(19) United States
(12) Patent Application Publication
(10) Pub. No.: US 2010/0286174 A1
(43) Pub. Date: Nov. 11, 2010

(54) INHIBITING GSNOR

(75) Inventors: Jonathan S. Stamler, Chapel Hill, NC (US); Howard A. Rockman, Chapel Hill, NC (US)

Correspondence Address: BACON & THOMAS, PLLC, 625 SLATERS LANE, FOURTH FLOOR, ALEXANDRIA, VA, 22314-1176, US

(73) Assignee: Duke University, Durham, NC (US)

(21) Appl. No.: 12/782,059

(22) Filed: May 18, 2010

Related U.S. Application Data
(63) Continuation-in-part of application No. PCT/US10/00762, filed on Mar. 12, 2010.
(60) Provisional application No. 61/161,458, filed on Mar. 19, 2009.

(51) Int. Cl. A61K 31/519; A61K 31/405; A61K 31/402; A61P 11/00; A61P 9/10; A61P 9/00; A61P 13/12

(52) U.S. Cl. 514/260.1; 514/419; 514/427

(57) ABSTRACT

Treatment of myocardial infarction to reduce infarct size and organogenesis is provided by administration of agents selected from the group consisting of

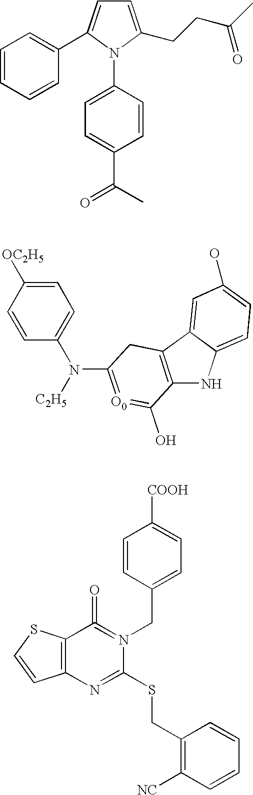

### CROSS-REFERENCE TO RELATED

This application is a continuation-in-part of PCT/US10/00762 which claims priority from U.S. Provisional Application No. 61/161,458, the whole of which is incorporated herein by reference.

### FIELD OF INVENTION

This invention is directed to enabled treatment of myocardial infarction with nitrosoglutathione reductase (GSNOR) inhibitors and to enabled organogenesis of failing heart, liver, kidney and lungs with GSNOR inhibitors.

### BACKGROUND OF INVENTION

Stamler et al. Publication No. 2008/0206738 (published Aug. 28, 2008) and Sanghahl et al. WO 2009/076665 A1 (published Jun. 18, 2009) mention modulation and/or inhibition of GSNOR but neither of these provides an enabled treatment of myocardial infarction or an enabled method of organogenesis of failing organs.

### SUMMARY OF INVENTION

This invention in a first embodiment is directed to treating a patient with myocardial infarction comprising administering to said patients agent selected from the group consisting of

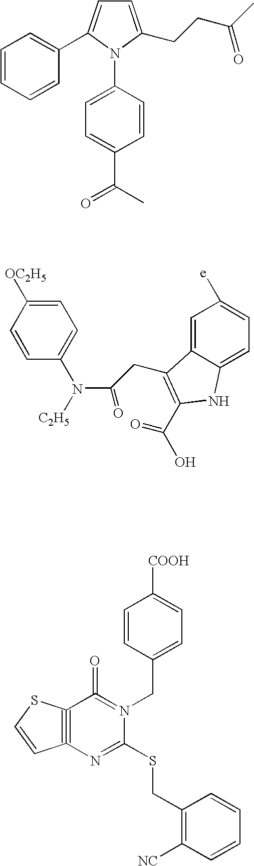

or a pharmaceutically acceptable salt or ester thereof in an amount effective to decrease infarct size (by at least 10% of infarcted myocardium compared to no treatment)

This invention in an off shoot of the first embodiment denoted the second embodiment is directed to administering agent selected from the group consisting of

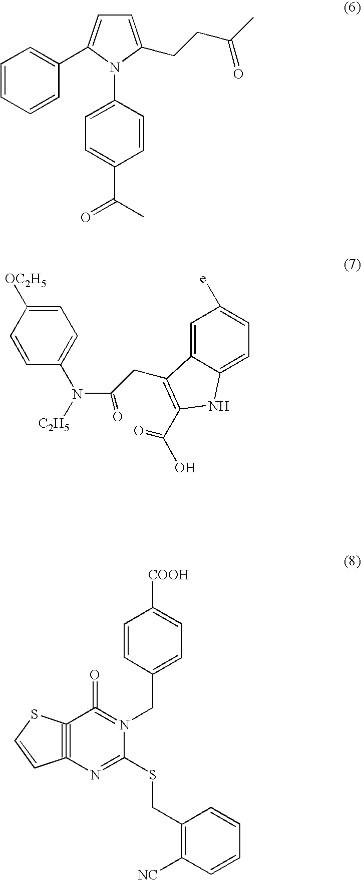

or a pharmaceutically acceptable salt or ester thereof to a patient with a failing organ in an amount to promote organogenesis by at least 10% (increase organ function by at least 10%).

### DETAILED DESCRIPTION

The three agents for the first and second embodiments are made as described in WO2009/07665 A1, the whole of which is incorporated herein by reference.

Said three agents for the first and second embodiments are administered by mouth or intravenously. The effective amount of each ranges from blood concentration ranging from 100 nanomolar to 100 micromolar, e.g. 5 to 20 micromolar, for at least_{—}1_d.

The patients treated in the second embodiment have, for example, heart failure, kidney failure, liver failure, or lung failure.

Background for the invention particularly showing genetic elimination of GSNOR −/− in mice is set forth in PNAS 106 (15) 6297-6302, pages 6297-6303 (Apr. 14, 2009), the whole of which is incorporated herein by reference.

Background and illustration of the invention herein is shown in the Background and Working Examples herein.

### BACKGROUND EXAMPLE 1

### Myocardial Infarct Size is Reduced in GSNOR −/− Mice Following Acute Coronary Ligation

To determine the effect of increased nitrosoglutathsone (SNO) bioavailability on myocardial response to ischemia, we ligated the left anterior descending (LAD) coronary artery of wild type (WT) and GSNOR −/− mice. Forty-eight hours following ligation, hearts were explanted and infused with trypan blue to demarcate the ischemic area susceptible to infarction, defined as the area at risk (AAR), and counterstained with triphenyltetrazolium chloride (TTC) to identify infracted regions within the AAR. Despite similar AARs between the groups, GSNOR −/− hearts demonstrated a significantly smaller proportion of infarction myocardium compared to WT mice (60±5% vs. 80±10% respectively; *, P=0.02). To rule out aberrant left coronary anatomy as the etiology of reduced infarct size in the GSNOR −/− mice, silicone casts were made of the mice hearts that revealed similar coronary artery anatomies.

### WORKING EXAMPLE I

60 y.o. white male presents with an elevated troponin, ST elevation and chest pain. He is treated with aspirin, beta blockers and compound 8 for 30 days in an amount to provide a blood concentration of said compound of 10 micromolar. His echo shows impaired wall motion in the anterior distribution. At 30 days his echo is normal.

### WORKING EXAMPLE II

Please supply prophetic example on patient with heart failure using compound 8. A 70 y.o with a history of repeated MI's and multiple admissions for heart failure, presents with class III symptoms. He is started on compound 8 (final concentration 10 micomolar), and over the next year his symptoms improve and he does not require an admission to the hospital.

### WORKING EXAMPLE III

A 26 yo with interstitial lung disease and resting shortness of breath is begun on compound 8 with symptomatic improvement. The patient improves on a 6 min walk test and breathes comfortably at rest.

### WORKING EXAMPLE IV

A 50 yo with diabetic nephropathy and creatinine of 3 is started on compound 6 at a final concentration of 6 micromolar and at follow up 2 months later, the creatinine is 2.

### Variation

Variation will be obvious to those skilled in the art. Therefore, the scope of the invention is defined by the claims.

What is claimed is:

1. A method of treating a patient with myocardial infarction comprising administering, to said patient agent selected from the group consisting of 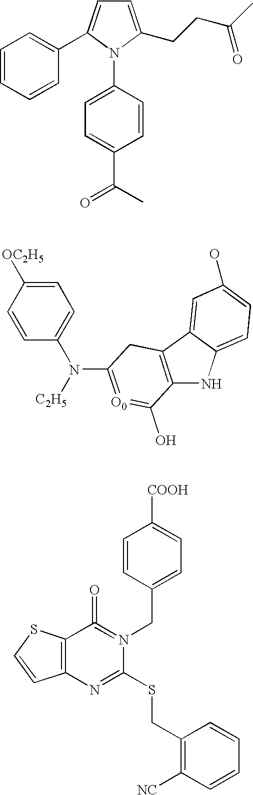
in an amount effective to decrease infarct size.

2. A method of treating a patient with organ failure comprising administering to said patient agent selected from the group consisting of 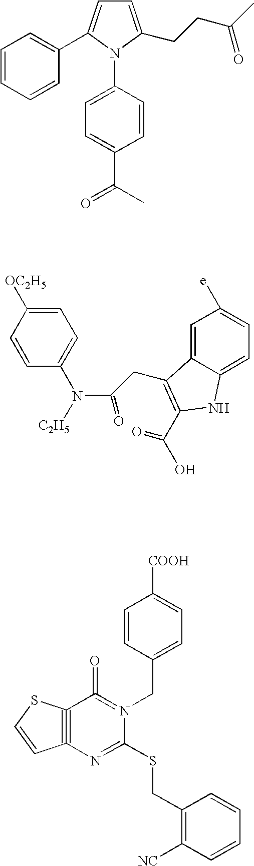
in an amount effective to increase organ function.

3. The method of claim 2 where the patient is afflicted with heart failure.

4. The method of claim 2 where the patient is afflicted with lung failure.

5. The method of claim 2 where the patient is afflicted with kidney failure.
